(19) United States
(12) Patent Application Publication
(10) Pub. No.: US 2024/0293350 A1
(43) Pub. Date: Sep. 5, 2024

(54) DEVELOPMENT OF AN INNOVATIVE INHALATION FORMULATION OF NAFAMOSTAT MESYLATE FOR THE MANAGEMENT OF COVID-19

(71) Applicants: EGE UNIVERSITESI, Izmir (TR); MEFAR ILAC SAN. A.S., Istanbul (TR)

(72) Inventors: Kambez HAJIPOURAN BENAM, Denver, CO (US); Ozlem YESIL, Izmir (TR); Tuncay GOKSEL, Izmir (TR); Mine OZYAZICI, Izmir (TR); Mesut ARICI, Izmir (TR); Aysu YURDASIPER, Izmir (TR); Ozlem GOKSEL, Izmir (TR)

(73) Assignees: EGE UNIVERSITESI, Izmir (TR); MEFAR ILAC SAN. A.S., Istanbul (TR)

(21) Appl. No.: 18/258,857

(22) PCT Filed: Dec. 22, 2021
(86) PCT No.: PCT/TR2021/051456

§ 371 Date: Jun. 22, 2023

(30) Foreign Application Priority Data
Dec. 22, 2020 (WO) PCT/TR2020/051347

(51) Int. Cl. A61K 31/245; A61K 9/00; A61K 47/12; A61K 47/18; A61K 47/20; A61K 47/26

(52) U.S. Cl. CPC A61K 31/245; A61K 9/0078; A61K 47/12; A61K 47/183; A61K 47/20; A61K 47/26

(57) ABSTRACT

The development of innovative inhalation formulations and preparation methods are provided to be used in the prophylaxis of COVID-19-related mortality from a broad-spectrum serine protease inhibitor NM, which has a potent anti-viral and anti-inflammatory potential. A drug for the infection treatment of SARS-COV-2, Influenza A, Influenza B and NL63 viruses includes the drug substance Nafamostat mesylate, which is a serine protease inhibitor in its inhalation form.

### CROSS REFERENCE TO THE RELATED APPLICATIONS

This application is the national phase entry of International Application No. PCT/TR2021/051456, filed on Dec. 22, 2021, which is based upon and claims priority to International Application No. PCT/TR2020/051347, filed on Dec. 22, 2020, the entire contents of which are incorporated herein by reference.

### TECHNICAL FIELD

The invention is related to the innovative inhalation formulation containing the drug substance nafamostat mesylate (NM) developed against COVID-19 disease and the method of preparing this formulation.

### BACKGROUND

The severe acute respiratory syndrome can be fatal in patients with Coronavirus 2 (SARS-CoV-2) and associated Coronavirus Disease 2019 (COVID-19) with rapid inappropriate excessive inflammatory response, severe pneumonia or acute coronary syndrome development.

It can present a severe, potentially life-threatening course in 5-10% of patients even though the majority of COVID-19 patients have a mild or moderate clinical course. There is no specific effective antiviral treatment for COVID-19 infection. Remdesivir, favipiravir, chloroquine, hydroxychloroquine, lopinavir/ritonavir are the most commonly used antiviral agents both in Turkey and in the world. In addition, nitazoxanide and ivermectin have recently been reported to have immunomodulatory activity against in vitro SARS-COV-2 (McCreary&Pogue, 2019; Beigel et al., 2020; Bergman, 2020). These known existing antiviral drugs provide a low level of protection against the disease. A drug development study for systemic disease prophylaxis is yet to be found in the literature even though there are many drugs understudy in the treatment of COVID-19.

In light of all this information, effective drugs are needed in disease management in order to calm the COVID-19 pandemic crisis, provide protection before the occurrence of the disease, or prevent the progression of severe forms of the disease in infected individuals.

Since NM is an important inhibitor of the serine protease, which contributes significantly to the development of airway inflammation, especially allergic airway inflammation, it is a very important anti-infection agent since it blocks the infection of the airway epithelial cells with the virus. In addition to the widespread use of NM with its therapeutic effect in acute inflammatory diseases such as intravascular coagulation, shock, pancreatitis, there are in vitro studies in the literature showing that it may also suppress SARS-COV-2 infection. Niemeyer et al. (2021) stated in their study targeting antiviral strategies in SARS-COV-2 replication and pathogenesis that nafamostat Mesylate (NM), a broad-spectrum serine protease inhibitor that blocks the host protease activation of viral spike protein, has both antiviral and anti-inflammatory effects. They emphasized in this context that dual therapeutic use may be advantageous in the treatment of SARS-COV-2 and other seasonal human coronaviruses. Si et al. (2021) stated that the duration of treatment was improved by applying Nafamostat and Oseltamivir to the airway chips after infection with the Influenza A virus in the microfluidic bronchial airway on-a-chip model they developed. Kirkpatrick & Millard (2021) stated that Nafamostat administered after infection with SARS-COV-2 decreased the viral load more than Remdesivir. Similarly, Bein et al. (2021) reported that Nafamostat was much more effective after infection with the NL63 virus than Remdesivir in the gut-on-a-chip model they developed.

It is possible that nafamostat mesylate (NM), which has many uses with similar mechanisms in different inflammatory diseases, is effective in the prophylaxis of systemic disease due to COVID-19, however, its use with this indication is not yet known.

NM is a synthetic pan-serine protease inhibitor approved for clinical use in Japan and South Korea since the late 1980s. However, NM is used as a rapid-acting proteolytic inhibitor administered intravenously during hemodialysis to prevent fibrinogen proteolysis and as an anti-inflammatory for pancreatitis (Akshintala et al., 2013; Choi et al., 2015).

It is predicted that NM can prevent the cell entry of SARS-COV-2 since the important amino acid sequence homology of S proteins of MERS-COV and SARS-COV-2 is shared (Lu 2020). NM has been shown to prevent cells from being infected with the virus in Simian Vero E6 cells infected with SARS-COV-2 in cell culture experiments (McKeea et al., 2020). Benam K. H. et al. reported that NM demonstrated antiviral activity in pre-clinical in-vitro lung models of the coronavirus infection using primary human mucociliary airway epithelial cells (Benam 2016, 2020). As a matter of fact, it has been shown in two studies that the entry of SARS-COV-2 into the cell is dependent on the spike (S) proteins of the virus, that the priming of this protein starts with the host cell proteases and that the intracellular entry it provides using ACE2 can be blocked by the serine protease TMPRSS2, which is a clinically effective protease inhibitor, and therefore there is a possible therapeutic value of serine proteases in SARS-COV-2 (Hoffmann 2020).

### SUMMARY

The primary object of the invention is to prevent the systemic spread of the virus in the prophylaxis and prevention the progression of severe disease in individuals infected with COVID-19, by developing the drug substance Nafamostat Mesylate (NM), which is a serine protease inhibitor, in the form of inhalation preparation.

SARS-COV-2 virus enters into the cell with the spike protein S, through the human lung airway epithelial cell transmembrane protease serine 2-TMPSS2 enzyme. With the use of NM, inhibition of the serine protease enzyme is provided and the penetration of the virus into the lower airway epithelium and its passage into the systemic circulation is prevented. Thus, a possible pneumonia or inappropriate excessive inflammatory response will be suppressed. With the NM inhalation preparation, the disease can be treated in individuals infected with COVID-19 in the early period before the virus becomes systemic.

The object of the invention is to prepare the drug containing the drug substance NM in the form of an inhalation, which provides transportation to the airways and alveoli by inhalation, and to prevent the development of severe disease or to prevent the prophylaxis of pneumonia in individuals infected with the virus. In addition, it is given for protective purposes to people at risk of transmission in order to prevent contamination between people. With the NM inhalation preparations developed within the scope of the invention, it is aimed to prevent the formation of rapid and inappropriate excessive inflammatory response in infected patients by activating prophylactic therapeutic mechanisms for the virus factor, and accordingly to reduce the risk of death.

There is no inhalation form of drug formulation even though there is a parenteral form of the drug substance NM. Drugs administered by inhalation have some advantages over drugs administered systemically. Inhalation treatment is the preferred treatment due to the direct effect on the target organ, less drug dose, faster effect and less side effects. Inhalation treatment in lung diseases has the advantage of applying the drug directly to the place of action. In addition, it is thought that the inhalation form will prevent viruses in the mouth, nose and upper respiratory tract from causing infection. Inhalation therapy in COVID-19 infection is a highly effective preventive treatment method because it has the advantage of a faster therapeutic effect. With the help of a nebulizer, sick individuals can practically administer the drug themselves without the help of a hospital or any healthcare personnel. Inhalation forms are in the presentation forms of lyophilized powder, suspension and solution.

With these inhalation preparations developed with the invention, it will be possible to use the drug quickly and practically in patients at the onset of virus infection.

The advantages and innovative aspects of the invention are listed below:
Developing a drug that can be used in an inhalation form to eliminate symptoms before severe disease occurs in COVID-19 patients, against the disease which is now seen as the number one problem in the world,
Contributing to scientific studies conducted on a national and international basis with these studies,
Bringing an important economic value to Turkey with this product to be developed,
Making a positive contribution to the current deficit of Turkey with the value to be provided,
Providing an output that will create an important prestige for Turkey in the world.

### DETAILED DESCRIPTION OF THE EMBODIMENTS

The invention is a drug for the infection treatment of SARS-COV-2, Influenza A, Influenza B and NL63 viruses and contains the drug substance Nafamostat mesylate, which is a serine protease inhibitor in its inhalation form.

Since nafamostat mesylate (NM) is an important inhibitor of the serine protease, which contributes significantly to the development of airway inflammation, especially allergic airway inflammation, it is a very important anti-infection agent since it blocks the infection of airway epithelial cells with the virus. In addition to the widespread use of NM with its therapeutic effect in acute inflammatory diseases such as intravascular coagulation, shock, pancreatitis, there are in vitro studies in the literature showing that it may also suppress SARS-COV-2 infection. Based on this, NM drug substance promises that it can be used as a prophylactic potential drug for SARS-CoV-2 infection. The accumulated cumulative clinical safety use information of NM also makes NM advantageous in terms of safety in the treatment of COVID-19 infection.

Within the scope of the invention, the systemic spread of the virus can be prevented in the prophylaxis of severe disease in individuals infected with COVID-19 by developing the drug substance nafamostat Mesylate (NM), which is a serine protease inhibitor, in the form of inhalation preparation. SARS-COV-2 virus enters into the cell with the spike protein S, through the human lung airway epithelial cell transmembrane protease serine 2-TMPSS2 enzyme. Inhibition of the serine protease enzyme will be ensured with the use of NM, and the penetration of the virus into the lower airway epithelium and its passage into the systemic circulation will be prevented. Thus, possible pneumonia or inappropriate excessive inflammatory response will be suppressed. With the NM inhalation preparation, the disease can be treated in individuals infected with COVID-19 in the early period before the virus becomes systemic.

Lyophilized powder formulations, characterization and performance tests of nafamostat mesylate for inhalation solution: Solutions of various excipients (osmotic agent, lyophilization/stabilization agent, pH adjusting agent, tonicity agent) were prepared with nafamostat mesylate. The pH range in which the solutions of the drug substance and the excipients prepared in water for injections or water for inhalation have the highest stability was determined. Various pH regulating agents, especially various organic acids, have been used for this purpose. Meanwhile, stabilization agent and pH adjusting agent concentrations were determined. The product in lyophilized form was developed after these formulation compositions were determined. The lyophilized powder product in the type 1 glass vial is applied together with a nebulizer after being dissolved/diluted with sterile water for injection. Drug substance quantification, pH, osmolality, moisture content, content uniformity, impurity and degradation products and sterility tests were performed on the prepared formulations.

Substances used in the preparation of lyophilized powder formulations of nafamostat mesylate for inhalation solution and their concentrations:
Nafamostat mesylate (0.01-10.0% w/v)
Mannitol (2.0-10.0% w/v)
Sodium edetate (0.0-0.20% w/v)
Sodium acetate (0.0-0.75% w/v)
Glycine (0.0-0.50% w/v)
Aspartic acid (0.0-0.5% w/v)
Acetic acid q.s. (pH adjustment)
Sodium hydroxide q.s. (pH adjustment)
Hydrochloric acid q.s. (pH adjustment)
Methanesulfonic acid q.s. (pH adjustment)
Sufficient amount of water for injections (q.s.)

### In Vitro/Ex Vivo Antiviral Activity Tests of the Drug Substance (NM) and Inhalation Formulations

Safety Tests with Ex Vivo PCLS Method

Safety tests of both drug substance and its inhalation formulations were performed using various doses with PCLS (Precision cut lung slices), which is an organotypic tissue culture technique in ex vivo pig lung tissue/organ. In this context, the pig, whose viability was preserved, was anesthetized with the appropriate euthanasia method and the lung tissue was directly obtained postmortem in order to preserve the viability of the tissue under sterile conditions. After being cannulated with a flexible catheter and filled with 1.5% low melting point agarose solution prepared in EMEM at 37° C., the lungs were separated into lobes on ice and processed as PCLS in sections, with a microtome device. PCLS were washed with DMEM containing 100 U/mL penicillin and 100 μg/mL streptomycin for 2-4 hours, and cultured in DMEM F12 medium containing sodium bicarbonate, 200 mM L-glutamine, 0.1 mM NEAA, 15 mM HEPES, 100 U/mL penicillin and 100 μg/mL streptomycin in cell culture vessels at 37° C., 5% CO₂, and for 1-6 days under humid conditions. The drug was administered with different doses on PCLSs, next to the control group which were not administered.

### Characterization Tests

### Determination of Cytokine for Determining the Immune Response

Supernatant samples were collected after drug administration to measure the amounts of extracellular cytokines in PCLS in order to determine the immune response, and PCLSs were lysed in PBS containing 1% TritonX-100 to measure intracellular cytokine amounts. After 0.2% proteinase inhibitor cocktail was added to the samples, cytokine contents (IFN-I, IL-1β, IL-1α, IL-6, IL-10, TNFα, MIP-2 and eotaxin) were tested in accordance with the kit instructions with the ELISA method.

### Viability Analyses

Quantitative measurement of cell viability after administration of both the drug substance and the inhalation formulations in ex vivo lung culture was examined by CCK-8 and WST-1 activity tests associated with mitochondrial activity in living cells. In addition, examination was made with LDH activity increased by cell membrane degeneration in culture supernatants. In addition, with Live&Dead cell viability kit and Propidium iodide fluorescent dye, the viability of ex vivo tissue was qualitatively imaged under fluorescent/confocal microscope.

### Measuring the Amount of Protein

The total protein concentration of PCLSs was performed with BCA test (BCA Protein Assay Kit, Pierce, Rockford, USA) within the manufacturer's instructions after administration of the drug substance and inhalation formulations.

### Immunofluorescence Staining

PCSLs were prepared in PBS and fixed for 4 hours with 4% Paraformaldehyde for the monitoring of dendritic cell markers on the cell surface (CD40, CD86 and CD11c antibodies with MHC class II mAbs). Stored at 4° C. in 0.01% sodium azide. Prepared in PBS after PBS washes and made permeable in 0.25% Triton X-100. Stained with primary antibody markers and incubated overnight at +4° C. in the dark, after waiting for 30 minutes for the blocking of non-specific bindings in PBS containing 10% BSA following PBS washing. Stained with compatible fluorescence-marked secondary antibodies (Alexa Fluor® 488, 647) at room temperature for 45 minutes the next day, after PBS wash. The nuclei of the cells were then stained with DAPI and sealed with a sealing solution (50% glycerol, 50%0.1 M NaHC03 in water, pH 7.4). Afterwards, the cells were imaged under inverted fluorescent/confocal microscope and the obtained images were analyzed in the ImageJ software.

What is claimed is:

1. A drug for an infection treatment of SARS-COV-2, Influenza A, Influenza B and NL63 viruses, comprising a drug substance nafamostat mesylate, wherein the drug substance nafamostat mesylate is a serine protease inhibitor in its an inhalation form.

2. The drug according to claim 1, wherein the inhalation form is in a form of lyophilized powder for an inhalation solution.

3. A method of preparing the drug according to claim 2, wherein the drug is prepared in the form of lyophilized powder for the inhalation solution containing the drug substance nafamostat mesylate.

4. The method according to claim 3, wherein an osmotic agent, a freeze-drying/stabilization agent, a pH adjusting agent, and a tonicity agent, are added together with the_drug substance in lyophilized powder formulations of nafamostat mesylate for the inhalation solution, wherein the osmotic agent, the freeze-drying/stabilization agent, the pH adjusting agent, and the tonicity agent are various excipients.

5. The method according to claim 4, wherein a pH is adjusted in a range of 3.5-5.5 when the lyophilized powder formulations of nafamostat mesylate for the inhalation solution are diluted.

6. The method according to claim 4, wherein an osmolality is adjusted in a range of 300-550 mOsm/kg when the lyophilized powder formulations of nafamostat mesylate for the inhalation solution are diluted.
